(19) United States
(12) Patent Application Publication
(10) Pub. No.: US 2003/0099695 A1
(43) Pub. Date: May 29, 2003

(54) Stabilised oversaturated transdermal therapeutical matrix systems

(76) Inventor: Walter Mueller, Neuwied (DE)

Correspondence Address: BIRCH STEWART KOLASCH & BIRCH, PO BOX 747, FALLS CHURCH, VA, 22040-0747, US

(21) Appl. No.: 10/221,650

(22) PCT Filed: Mar. 5, 2001
(86) PCT No.: PCT/EP01/02449

(30) Foreign Application Priority Data
Mar. 16, 2000 (DE) 100 12 908.0

(51) Int. Cl. A61K 9/70

(52) U.S. Cl. 424/449

(57) ABSTRACT

A transdermal therapeutic system of the matrix type, comprising an active substance-impermeable backing layer, a detachable protective layer and an active substance-containing matrix based on hydrophobic polymers, the active substance having a melting point above room temperature and being present at least during part of the application time of the TTS in a concentration exceeding the saturation solubility, is characterized in that a polyacrylate polymer is admixed to the hydrophobic base polymers of the active substance matrix, or/and in that the matrix layer containing the hydrophobic polymers is provided with a self-adhesive skin-contact layer based on polyacrylates.

The invention relates to transdermal therapeutic systems (TTSs) of the matrix type comprising an active substance-containing matrix based on hydrophobic polymers. More particularly, the invention relates to TTSs of the type mentioned which are at least temporarily oversaturated with active substance and wherein measures have been taken to prevent the recrystallization of an active substance which is solid at room temperature.

The invention furthermore relates to processes for the production of transdermal therapeutic systems of the type mentioned.

Transdermal therapeutic systems (TTSs) are relatively new medicinal forms but meanwhile have become quite established in a variety of application fields. Their general advantages lie in preventing the so-called first-pass effect and in maintaining therapeutically useful plasma levels over a period of up to 7 days. The application possibilities of a transdermal system are, however, frequently restricted by the fact that they are mainly suitable for administering drugs which are very potent and are already effective in very small doses. The reason for this lies in the barrier properties of the stratum corneum of the skin, which limit or prevent the absorption of drugs via the skin.

For this reason, a considerable effort has been made to at least partially by-pass this obstacle. This can be achieved, for example, by employing permeation enhancers (also called penetration enhancers), which weaken the skin's barrier action. Furthermore, a sufficient active substance flow through the skin can also be attained by actively transporting the active substance by means of electric current. A further measure through which the absorption of active substances through the skin can be promoted consists in aiming at a thermodynamic activity of the active substance in the transdermal therapeutic system which is as high as possible.

Permeation enhancers are substances which affect the stratum corneum in such a way that its diffusion resistance is reduced, thus increasing the transdermally administerable amount of active substance. A large number of substances are suitable as permeation enhancers, for example, fatty acids, fatty alcohols, dimethyl sulfoxide, partial glycerides and propylene glycol.

Transdermal systems enabling an active transport of the active substance are known as so-called electrophoresis or iontophoresis systems. Such systems have so far been employed first of all for transdermal application of predominantly topically active drugs. Recently, efforts are being made, however, which focus on minimizing the size of those systems for practical use so as to render them suitable for application of systemically active drugs too.

With the exception of the electrophoresis or iontophoresis systems described above, the active substance release of transdermal therapeutic systems is in principle based on the principle of passive diffusion of the active agent from the patch into and through the stratum corneum of the skin, and subsequent systemic absorption of the active substance.

The third above-mentioned possibility of improving the active substance uptake via the skin consists in rendering the thermodynamic activity of the active substance in the transdermal therapeutic system as high as possible. In this way it is possible to increase the flow of active substance. A very high thermodynamic activity is achieved if the active substance concentration of the active substance dissolved in the active substance-containing components of the TTS corresponds to the saturation concentration of the active substance concerned. Such TTSs, in addition, possess good storage stability.

A further increase of the thermodynamic activity of the active substance can be attained by raising the concentration of the active substance above its saturation concentration. However, the advantage of higher thermodynamic activity is linked to the disadvantage of such TTSs being physically unstable, i.e. the storage stability of such oversaturated systems is reduced.

The adverse effect on the storage stability is based on the fact that active substances which at room temperature are present in a solid state have a tendency to recrystallize in such oversaturated TTSs. Owing to the crystal growth or formation of crystals, the concentration of dissolved active substance decreases, with the consequence that the thermodynamic activity of the active substance is reduced and the release rate of the active substance lowered. It is for this reason that it is not possible to produce oversaturated TTSs containing partially undissolved active substance, as in such cases, due to the crystal growth, the concentration of the dissolved active substance will correspond to the saturation concentration already after a very short time.

There are, however, special TTS formulations where the state of oversaturation occurs only after application of the TTS to the skin, so that, prior to application, the storage stability is not adversely affected. Such systems reach the state of oversaturation by the fact that a solubilizer contained in the patch is likewise released from the system to the skin, respectively by the fact that the uptake of moisture from the skin reduces the saturation solubility of the active substance in the TTS. The advantage of such systems is their storage stability with respect to recrystallization. However, in these cases, too, the active substance must be prevented from quickly recrystallizing to a considerable extent during the application time of the TTS. This would make it impossible to achieve a sufficient active substance release during the intended duration of application.

The simplest way to produce TTSs that reach an oversaturated state during the application period is to base them on polysiloxanes. Polysiloxanes have only a very poor solubility for most active substances. To be able to load the polysyloxane matrices of such TTSs with sufficient amounts of dissolved active substance, it is necessary to add solvents to the polysiloxanes. Here, those solvents are used with preference which possess only restricted miscibility with the polysiloxanes and are present in the matrix in dispersed form, as droplets. In this way it is possible to largely prevent an adverse effect on the physical properties of the active substance matrix. The dispersed solvent droplets at the same time contain the predominant portion of the pharmaceutical active agent, which is why they can be regarded as micro-reservoirs for active substances.

Suitable and physiologically safe solvents are, for instance, propylene glycol, 1,3-butanediol, dipropylene glycol, tetrahydrofurfuryl alcohol and diethyleneglycolmonoethyl ether. These solvents are likewise absorbed trough the skin, whereby the solvent content in the active substance-containing TTS matrix is reduced. At the same time, the water released by the skin concentrates in the solvent droplets since the polysiloxanes can absorb water only to a very limited degree, owing to their extremely hydrophobic properties. Both mechanisms lead to oversaturation of the system (TTS) with active substance, in conjunction with an increased active substance flux through the skin. It must be observed, however, that this oversaturated state needs to be stabilised over a prolonged period of the application time.

Stabilizing the oversaturated state during the application time is important, in particular, because it was surprisingly found that in active substance-oversaturated TTSs with hydrophobic matrix formulation, recrystallisation of the active substance can not only occur in the matrix itself but also in a thin moisture film which can form during the application time between the active substance-releasing side of the TTS and the skin surface underneath.

Since the active substance is not absorbed by the skin as quickly as it is released from the TTS, this moisture film, too, is oversaturated with active substance. As a consequence the active substance can at least partially recrystallize during the application time in the area of this moisture film, which puts an end to the oversaturated state and reduces the thermodynamic activity of the active substance. This means that the thermodynamic activity of the active substance in the moisture film located immediately above the skin is lowered compared to the conditions existing in the matrix.

The active substance uptake from the TTS through the skin is thereby reduced, and the theoretic advantages of an active substance-oversaturated matrix are lost. In the polymeric matrix layers themselves, the tendency for recrystallisation is relatively weak due to the diminished diffusion coefficient and the generally inhibiting action of polymers on the formation of crystal nuclei.

The problem underlying the present invention was thus to stabilise the oversaturated state in TTSs of the matrix type which are based on hydrophobic polymers as matrix formulations and which are present at least during a part of the application time in the oversaturated state, in such a way that the oversaturated state is also maintained during a prolonged period of the application time. More particularly, the problem consisted in preventing that the active substance undergoes recrystallisation after its release from the TTS and before it is absorbed through the skin.

It was now surprisingly found that in active substance-oversaturated, hydrophobic polymer-based matrix TTSs having the features mentioned in the introductory portion of Claim 1, stabilisation of the oversaturated state during the application time is achieved by admixing a polyacrylate polymer to the hydrophobic base polymer(s) of the active substance matrix, or/and by providing the matrix layer containing the hydrophobic polymers with a self-adhesive skin-contact layer based on polyacrylates.

By means of the measures proposed in Claim 1, the formation of the moisture film mentioned above is prevented or suppressed and the risk of active substance recrystallisation occurring in the area between the active substance-releasing side of the TTS and the skin is reduced or eliminated. In this way, the thermodynamic activity of the active substance remains on a high level in such a TTS over a prolonged period of time (which is why those TTSs are called “stabilized”).

This in turn has the consequence that the TTS is able to deliver the active substance or active substances in therapeutic doses over a prolonged period of time, and that thereby the application time of the TTS, during which sufficient release rates must be achieved, is prolonged.

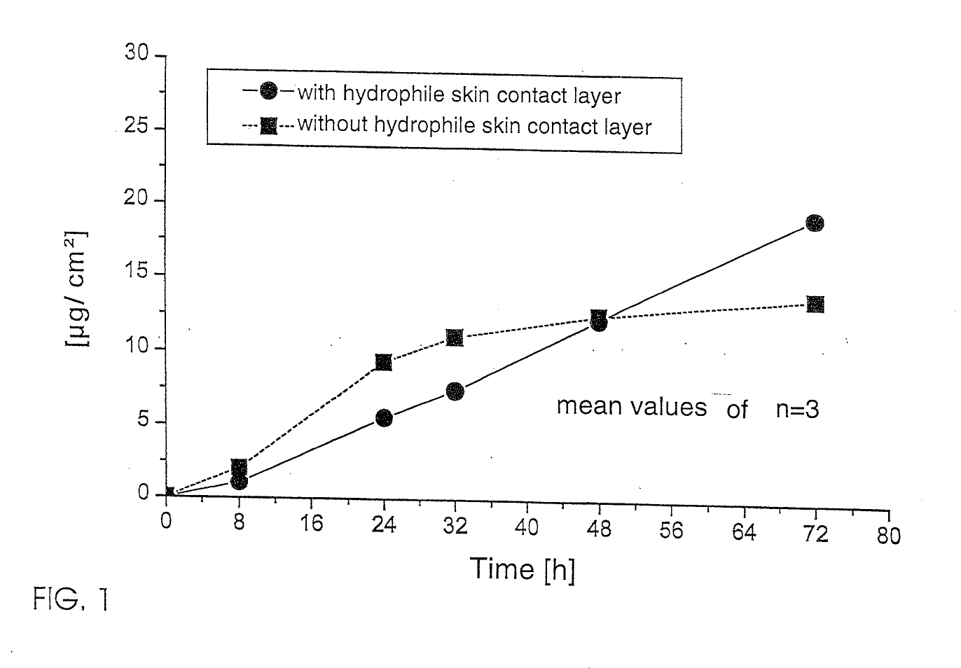

With the stabilised TTSs proposed by the invention it is, in particular, possible to maintain a largely constant release of active substance during the application time. This was proved by permeation studies with the Examples 1 to 3; the results are shown in FIGS. 1 to 3.

This results in further advantages such as improved or facilitated application as a consequence of the prolonged application time, higher therapy safety through stabilization of the delivery behaviour, as well as more efficient use of active substance. By improving the active substance release, the present invention further affords the possibility of broadening the range of applications of transdermal systems which are based on passive diffusion. In addition, the invention enables the manufacture of transdermal systems which can have a smaller surface area due to the high active substance release rates which can be achieved with the invention; this in turn is of advantage in manufacture and application.

The present invention is applicable for TTSs of the matrix type (matrix TTSs) whose active substance-containing matrix is made on the basis of hydrophobic polymers. The stabilizing effect achieved by the additional use of polyacrylates in principle comes to fruition in all active substance-oversaturated hydrophobic matrices.

In particular, the invention is of advantage in such TTSs which reach an oversaturated state in respect of the active substance only after application to the skin by way of absorption of moisture or by way of the release of solvents.

The structure of the TTSs according to the invention comprises an active substance-impermeable backing layer and a releasable protective layer to be removed prior to application, apart from the mentioned active substance-containing matrix.

In the simplest case, the active substance matrix of the systems according to the invention has a single-layer structure and is self-adhesive. But the invention also relates to TTSs of a more complicated configuration which have multilayered active substance matrices; in this case not all of the layers of the matrix have to be adhesive. In addition, the systems according to the invention may in special cases also contain a special control membrane which on account of its thickness and/or composition puts an upper limit on the active substance release.

In the TTSs according to the invention, polysiloxanes, preferably self-adhesive polysiloxanes, or polyisobutylene, polyisoprene, or a styrene-diene-styrene block copolymer, or mixtures of such hydrophobic polymers are preferably used as hydrophobic polymers which constitute the base polymers of the active substance matrix. Among the polysiloxanes, amine-resistant polysiloxanes are especially preferred.

According to the invention, the stabilised TTSs contain a polyacrylate which is admixed to the hydrophobic matrix layer, and/or an additional skin-contact layer which is superimposed on the hydrophobic matrix layer and is manufactured on the basis of polyacrylate adhesives.

The polyacrylates used are polymers which possess more or less hydrophile properties, depending on the monomers used. The portion of the polyacrylate polymer admixed to the hydrophobic matrix is preferably 40%-wt. at the most, relative to the total matrix. A still higher polyacrylate portion would lead to the properties of the active substance matrix being excessively determined by the polyacrylate. To achieve the effect according to the present invention—i.e. the reduction of the tendency towards formation of a moisture film and thus also of the tendency towards recrystallization—in a degree which is at least sufficient, the amount of the polyacrylate should be at least about 10%-wt., better still at least about 15%-wt., relative to the matrix layer. The admixed polyacrylate may also be a self-adhesive polyacrylate; if the polyacrylate is admixed to a self-adhesive matrix layer it does not need to be adhesive itself.

It is in principle sufficient to admix a hydrophile polyacrylate polymer at least to the matrix layer which is near the skin, i.e. the matrix layer which is in contact with the skin (skin-contact layer). This particularly applies to TTSs with multi-layered active substance matrices. The portion of the polyacrylate should amount to at least approx. 10%-wt., better still at least approx. 15%-wt., relative to the skin-contact layer, but maximally 40%-wt. relative to the total matrix.

Apart from polyacrylates it is also possible to use mixtures of polyacrylates with other hydrophile polymers, which are, in accordance with the invention, admixed to the hydrophobic base polymers(s) of the matrix or used to produce an additional skin-contact layer. As further hydrophile polymers, polyvinyl pyrrolidone and copolymers of the vinylpyrrolidone with vinyl acetate can be employed for instance.

Even where, as described above, mixtures of polyacrylates(s) with other hydrophile polymers are employed, the total portion of the hydrophile polymers admixed to the hydrophobic matrix should not exceed a value of 40%-wt., relative to the total matrix.

The polyacrylate itself, which is used in accordance with the present invention, may be a copolymer of any acryl and methacryl derivatives and vinyl compounds suitable for the purpose. The following monomers are mentioned by way of example: acrylic acid, methacrylic acid, acrylic acid ethyl ester, acrylic acid butyl ester, acrylic acid octyl ester, 2-ethylhexyl acrylate, 2-hydroxyethyl acrylate and vinyl acetate.

If the hydrophobic polymers-containing matrix layer is provided with a self-adhesive skin-contact layer based on polyacrylates, the thickness of this layer or film should be markedly smaller than that of the hydrophobic matrix layer(s). More particularly, the thickness of the said layer should not exceed a value corresponding to 50% of the thickness of the hydrophobic matrix layer(s), since otherwise the properties of the additional hydrophilic skin-contact layer—which likewise contains active substance—would dominate the properties of the system.

In a specific embodiment of the invention, the self-adhesive, hydrophilic skin-contact layer is a mixture of a self-adhesive polyacrylate and a hydrophilic polymer, preferably a film-forming polymer. As a hydrophilic film-forming polymer it is possible to use, for example, polyvinyl pyrrolidone or a copolymer of vinylpyrrolidone and vinyl acetate.

The manufacture of the inventive stabilised TTSs can be accomplished such that initially a solution is prepared which contains the hydrophobic base polymers and the admixed acrylate polymers, as well as, possibly, auxiliary substances, in a suitable solvent. The active substance is added to this matrix polymer solution and dissolved. If necessary, the active substance can be added in dissolved form, possibly using solvents suitable specifically for this active substance. The active substance-containing matrix polymer mass obtained is then coated on a suitable film and subjected to drying or to a heat treatment to remove the solvents of the polymers. The dried matrix layer is covered with a further suitable film, and subsequently the individual TTSs are punched from this laminate.

The advantage of the method described above consists in that no additional coating process is necessary in order to provide the TTS with stabilising properties.

In the manufacture of stabilised TTSs according to the invention which are characterized by a self-adhesive hydrophile skin-contact layer based on polyacrylates, the hydrophobic, active substance-containing matrix layer and the hydrophile skin-contact layer are produced in separate coating processes. The individual layers are subsequently laminated onto each other, which yields the complete system.

Here, the skin-contact layer can be loaded with active substance already during the manufacture, or it can be manufactured free of active substance. In the latter case the active substance enters the skin-contact layer by way of diffusion from the hydrophobic matrix layer after the laminate has been prepared.

The invention will be explained in the following by way of examples.

### EXAMPLE 1

### TTS Comprising Estradiol

### EXAMPLE 1a

### TTS Without Hydrophile Skin-Contact Layer

### COMPARISON EXAMPLE 1

1.0 g of estradiol hemihydrate were dissolved in 22.75 g of 1,3-butanediol, and the solution was thickened by adding 0.7 g of hydroxypropyl cellulose. Then, 60 g of a solution of an amine-resistant polysiloxane adhesive (BIO-PSA 4301; Dow-Corning; solids content: 70%-wt.) were added to this solution, and the active substance solution was dispersed in the solution of the adhesive by stirring.

Subsequently, the mass is coated, using an Erichson doctor knife, in a thickness of 200 μm onto a film which has been rendered abhesive (Scotchpak 1022; 3M), and dried for 20 min at 40° C. This yields a matrix film with a coating weight of 120 g/m². The dried matrix film is covered with the backing layer of the TTS (Scotchpak 1220; 3M).

### EXAMPLE 1b

### TTS With Hydrophile Skin-Contact Layer

1.0 g of estradiol hemihydrate are dissolved in 20.0 g of 1,3-butanediol, and subsequently 20.0 g of a Kollidon 90F solution (Kollidon 90F is a polyvinyl pyrrolidone; BASF) having a solids content of 25%-wt. are added while stirring.

Thereafter, 145 g of a solution of a polyacrylate adhesive (Durotak 387-2287; National Starch & Chemical; solids content: 51%-wt.) are added, and the mixture is homogenized by stirring. The mass is coated in a thickness of 50 μm onto a film which has been rendered abhesive (Scotchpak 1022; 3M), and is dried at 40° C. for 15 min.

The dried film has a coating weight of 16 g/m².

Then, the abhesive-rendered film is removed from the hydrophobic matrix layer prepared under 1a, and the matrix layer is laminated onto the skin-contact layer.

The finished TTSs are then punched out from this total laminate.

The results of a comparative permeation study between samples without skin-contact layer (1a) and samples with hydrophile skin contact layer (1b) are represented in FIG. 1.

### EXAMPLE 2

### Transdermal System (TTS) With Estradiol

### EXAMPLE 2a

### TTS Without Hydrophile Skin-Contact Layer

### COMPARISON EXAMPLE 2

5.0 g of estradiol hemihydrate are dissolved in 38.5 g ofdipropylene glycol. To this solution are added 124 g of a solution of a polysiloxane adhesive (BIO-PSA 4301; Dow-Corning; solids content: 70%-wt.), and the active substance solution is dispersed in the adhesive solution while stirring.

Thereafter, the mass is coated by means of an Erichson doctor knife onto a suitable, film which has been rendered abhesive (Scotchpak 1022; 3M), and the solvent of the adhesive is removed by drying for 20 minutes at 45°. The dried film having a coating weight of 80 g/m² is then covered with a suitable film (e.g. Scotchpak 1220; 3M).

### EXAMPLE 2b

### TTSs With Hydrophilic Skin-Contact Layer

1.0 g of estradiol hemihydrate are dissolved in 10.0 g of dipropylene glycol, and subsequently 20.0 g of a Kollidon 90F solution (Kollidon 90F is a polyvinyl pyrrolidone) having a solids content of 25%-wt. are added while stirring.

Thereafter, 164 g of a solution of a polyacrylate adhesive (Durotak 387-2287; National Starch & Chemical; solids content: 51%-wt.) are added, and the mixture is homogenised while stirring. The mass is coated in a thickness of 50 μm with an Erichson doctor knife onto a film which has been rendered abhesive (Scotchpak 1022; 3M), and dried at 40° C. for 15 min. The dried film has a coating weight of 15 g/m².

The abhesive-rendered protective film is removed from the hydrophobic matrix layer prepared under 2a, and the said matrix layer is laminated onto the skin-contact layer.

The TTSs are then punched out of this total laminate.

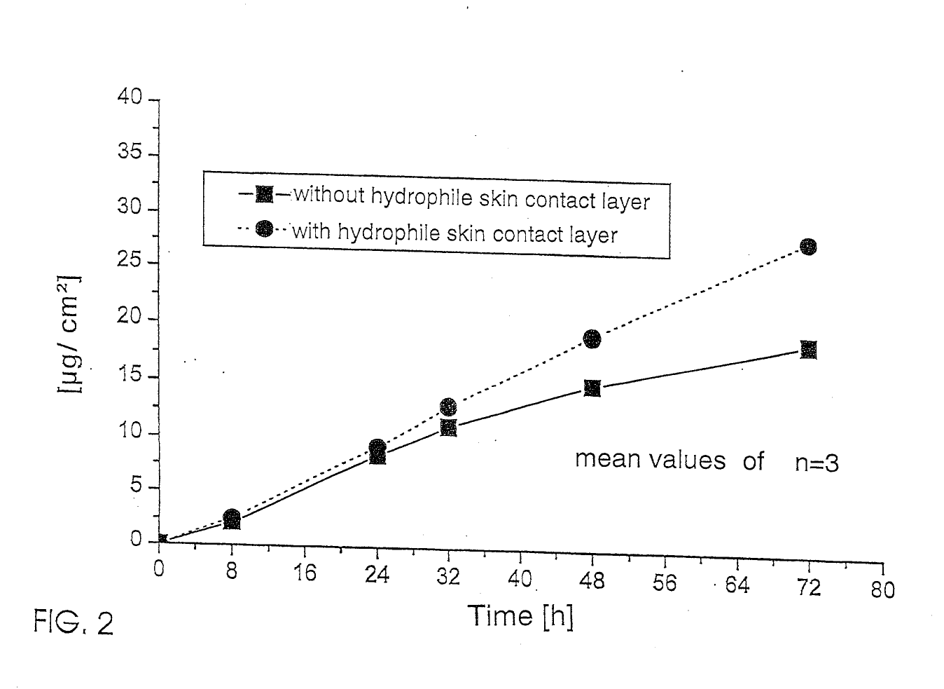

The results of a comparative permeation study between samples without skin-contact layer (2a) and samples with hydrophile skin-contact layer (2b) are represented in FIG. 2.

### EXAMPLE 3

### Monolithic Transdermal System (TTS) Based on Silicone Adhesives With Hydrophile Additives

1.2 g of estradiol hemihydrate are dissolved in 9 g of dipropylene glycol, and the solution is thickened by addition of 0.26 g of hydroxypropyl cellulose (Klucel NF). To this solution are added 88.0 g of silicone adhesive (BIO-PSA 4301; Dow-Corning; solids content: 70%-wt.), 10.0 g of a polyacrylate adhesive (Durotak 387-2287; solids content 51%-wt.; National Starch) and 1.2 g of a solution of Kollidon 90F in ethanol (solids content 25%-wt), and the mass is mixed while stirring.

The mass is coated in a thickness of 250 μm onto a film which has been rendered abhesive (Scotchpak 1022; 3M), using an Erichson doctor knife, and is dried for 15 min at 40° C. The dried film having a coating weight of 115 g/m² is then covered with a suitable film (e.g. Scotchpak 1220; 3M), and the finished patches are punched out of the total laminate.

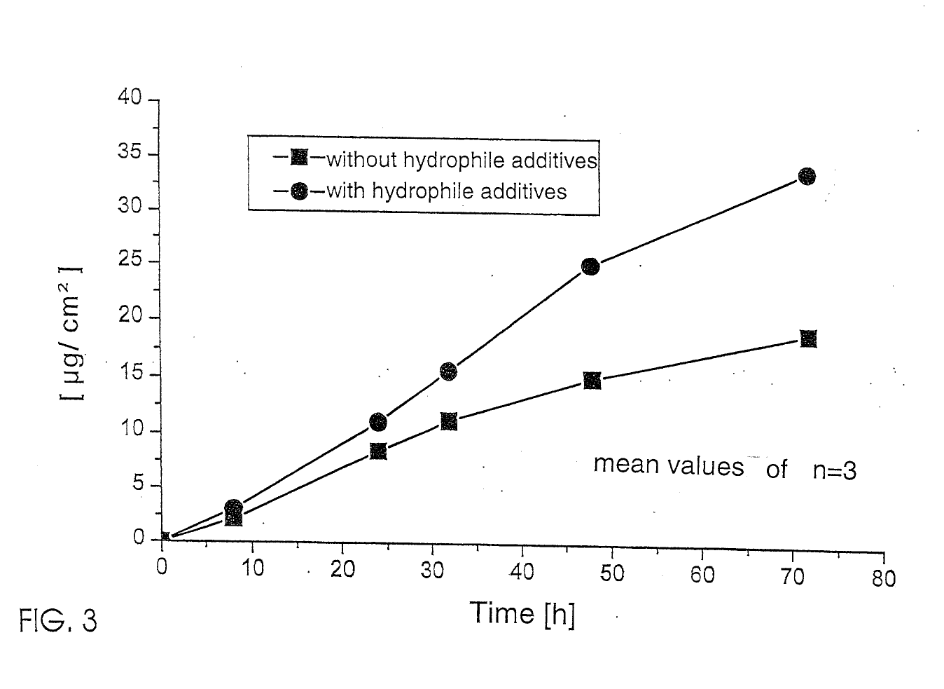

Example 2a serves as a comparison example. The results of a comparative permeation study between samples without hydrophilic additives (2a) and samples with hydrophilic additives (3) are represented in FIG. 3.

Permeation studies involving the systems prepared according to Examples 1 to 3.

The results of the comparison measurements are represented in FIGS. 1 to 3. These measurements were made using Franz diffusion cells and human epidermis. Each point is the mean of 3 independent measurements.

The time course of the permeation in FIGS. 1 to 3 clearly shows that in the case of the TTSs according to the present invention a constant release rate, and thus a stabilisation, is achieved for a period of at least 72 h, whereas in the case of the comparison examples a marked flattening of the permeation profile can be seen already after 32 h.

1. Transdermal therapeutic system of the matrix type, comprising an active substance-impermeable backing layer, a detachable protective layer and an active substance-containing matrix based on hydrophobic polymers, the active substance having a melting point above room temperature and being present at least during part of the application time of the TTS in a concentration exceeding the saturation solubility, characterized in that a polyacrylate polymer is admixed to the hydrophobic base polymers of the active substance matrix, or/and in that the matrix layer containing the hydrophobic polymers is provided with a self-adhesive skin-contact layer based on polyacrylates.

2. Transdermal therapeutic system of the matrix type according to claim 1, characterized in that the active substance matrix comprises as hydrophobic base polymers: polysiloxanes, preferably self-adhesive polysiloxanes, or polyisobutylene, polyisoprene or a styrene-diene-styrene block copolymer, or mixtures of such hydrophobic polymers; amine-resistant polysiloxanes being particularly preferred.

3. Transdermal therapeutic system of the matrix type according to claim 1 or 2, characterized in that at least the matrix layer coming into contact with the skin comprises a portion of a polyacrylate polymer, this portion preferably being at least 10%-wt., more preferably 15%-wt, relative to the skin contact layer, but maximally 40%-wt. relative to the total matrix.

4. Transdermal therapeutic system of the matrix type according to one of the preceding claims, characterized in that a polyacrylate is admixed to the hydrophic base polymer(s) of the active substance matrix, the total portion of the polyacrylate admixed to the hydrophic base polymer preferably being at least 10%-wt., more preferably 15%-wt., but maximally 40%-wt, each relative to the total matrix.

5. Transdermal therapeutic system of the matrix type according to one of the preceding claims, characterized in that, in addition, polyvinyl pyrrolidone or a copolymer of polyvinyl pyrrolidone and vinyl acetate is/are admixed to the hydrophobic base polymer(s) of the active substance matrix or at least to the matrix layer which is near the skin, the total portion of the polymers admixed to the hydrophobic base polymer preferably being at least 10%-wt., more preferably 15%-wt., but maximally 40%-wt., each relative to the total matrix.

6. Transdermal therapeutic system of the matrix type according to claim 1 or 2, characterized in that the skin-contact layer is a mixture of a self-adhesive polyacrylate and a hydrophile polymer, preferably a film-forming hydrophile polymer.

7. Transdermal therapeutic system of the matrix type according to claim 6, characterized in that the film-forming hydrophile polymer is polyvinyl pyrrolidone or a copolymer of vinylpyrrolidone and vinyl acetate.

8. Transdermal therapeutic system of the matrix type according to one or more of the preceding claims, characterized in that the system reaches an oversaturated state in respect of the active substance only after the system is applied to the skin, by way of absorption of moisture or by way of release of solvents.

9. Process for the production of transdermal therapeutic systems of one or more of claims 1 to 5 and 8, comprising the following steps:
a) Preparing a solution containing the hydrophobic base polymers and the admixed acrylate polymers;
b) adding and dissolving the active substance in the matrix polymer solution, or adding an active substance solution and mixing the same with the matrix polymer solution;
c) coating this mass on a film;
d) removing the unwanted solvents by heating or drying;
e) covering the dried matrix layer with a film;
f) punching out individual transdermal therapeutic systems.

10. Process for the production of a transdermal therapeutic system of the matrix type comprising a hydrophile skin-contact layer, according to one of claims 1, 2, 6 to 8, characterized by the following steps:
a) Preparing a solution or dispersion containing a hydrophobic base polymer and an active substance;
b) coating this polymer- and active substance-containing mass in a thin layer onto a film which has been rendered abhesive, and subsequent drying;
c) producing a second layer—the skin-contact layer—according to steps a) and b), the coating mass used containing an acrylate polymer and possibly in addition a hydrophile film-forming polymer, or a hydrophobic polymer mixed with an acrylate polymer and possibly in addition with a hydrophile film-forming polymer;
d) detaching the abhesive film from the matrix layer obtained in step b);
e) laminating the matrix layer obtained in step b) onto the skin-contact layer obtained in step c);
f) punching out individual transdermal therapeutic systems.
